(19) United States
(12) Patent Application Publication
(10) Pub. No.: US 2018/0133009 A1
(43) Pub. Date: May 17, 2018

(54) Installation of Cinching Cords and Annulus Rings onto a Cardiac Valve Annulus

(71) Applicant: Cardiac Implants LLC, Tarrytown, NY (US)

(72) Inventor: David ALON, Zichron Yaakov (IL)

(21) Appl. No.: 15/349,198

(22) Filed: Nov. 11, 2016

(51) Int. Cl. A61F 2/24

(52) U.S. Cl. CPC A61F 2/2445; A61F 2230/0006; A61F 2220/0016; A61F 2/2466

(57) ABSTRACT

This application discloses a variety of approaches for installing a cinching cord onto a cardiac valve annulus and for positioning an annulus ring onto a cardiac valve annulus. One aspect of the invention is directed to an apparatus for cinching a cardiac valve annulus. This apparatus comprises a catheter and a ring of material configured for delivery to the vicinity of a cardiac valve annulus via the catheter. The ring has an external surface. A plurality of whisker-like are barbs disposed on the external surface of the ring, and the barbs are configured to engage tissue when pressed against the tissue. The barbs are also configured to hold the ring in place until tissue ingrowth strengthens a bond between the ring and the annulus.

### BACKGROUND

U.S. application Ser. No. 14/895,711 (filed on Dec. 3, 2015), which is incorporated herein by reference in its entirety, discloses a variety of approaches for installing a cinching cord onto a cardiac valve annulus (e.g. the mitral valve annulus or the tricuspid valve annulus). After the cinching cord is installed and after tissue healing has strengthened the bond between the cinching cord and the annulus, the cinching cord can be used to reduce the size of the annulus. U.S. application Ser. No. 14/895,711 also discloses a variety of approaches for positioning an annulus ring onto a cardiac valve annulus, which is useful to prevent the annulus from expanding and/or to serve as a dock into which a replacement valve can be installed.

### FIELD OF THE INVENTION

This application discloses a variety of alternative approaches for installing a cinching cord onto a cardiac valve annulus and for positioning an annulus ring onto a cardiac valve annulus.

### SUMMARY OF THE INVENTION

One aspect of the invention is directed to a first apparatus for cinching a cardiac valve annulus. This apparatus comprises a catheter and a ring of material configured for delivery to the vicinity of a cardiac valve annulus via the catheter. The ring has an external surface. A plurality of whisker-like are barbs disposed on the external surface of the ring, and the barbs are configured to engage tissue when pressed against the tissue. The barbs are also configured to hold the ring in place until tissue ingrowth strengthens a bond between the ring and the annulus.

In some embodiments of the first apparatus, the shape of the ring is pre-formed to match the shape of the annulus onto which it will be installed.

In some embodiments of the first apparatus, the ring is formed from a shape-memory material that automatically returns to a predetermined shape after the ring exits the catheter.

In some embodiments of the first apparatus, the ring has a hollow core, and the apparatus further comprises a tether that runs through the hollow core.

In some embodiments of the first apparatus, the external surface of the ring includes a coating that promotes tissue ingrowth.

### BRIEF DESCRIPTION OF THE DRAWINGS

FIGS. 1A-1C depict a first embodiment of a cinching mechanism that is installed on a cardiac valve annulus in successive stages of deployment.

FIGS. 2A-D depict another embodiment of a cinching mechanism that is installed on a cardiac valve annulus in successive stages of deployment.

FIG. 3 depicts one embodiment of a cinching ring that can be delivered to a valve annulus via a catheter.

FIG. 4 depicts another embodiment of a cinching ring that can be delivered to a valve annulus via a catheter.

FIG. 5 depicts another embodiment of a cinching ring that can be delivered to a valve annulus via a catheter.

FIG. 6 depicts one embodiment of an annulus ring that has been installed onto the mitral valve annulus. 4

FIG. 7 depicts a variation of the FIG. 6 embodiment.

FIG. 8A depicts another embodiment of an annulus ring that has been installed onto the mitral valve annulus.

FIG. 8B is a detail of the FIG. 8A embodiment.

FIG. 9A depicts one alternative approach for loosely positioning an annulus ring in the vicinity of the mitral valve.

FIG. 9A depicts another alternative approach for loosely positioning an annulus ring in the vicinity of the mitral valve.

### DESCRIPTION OF THE PREFERRED EMBODIMENTS

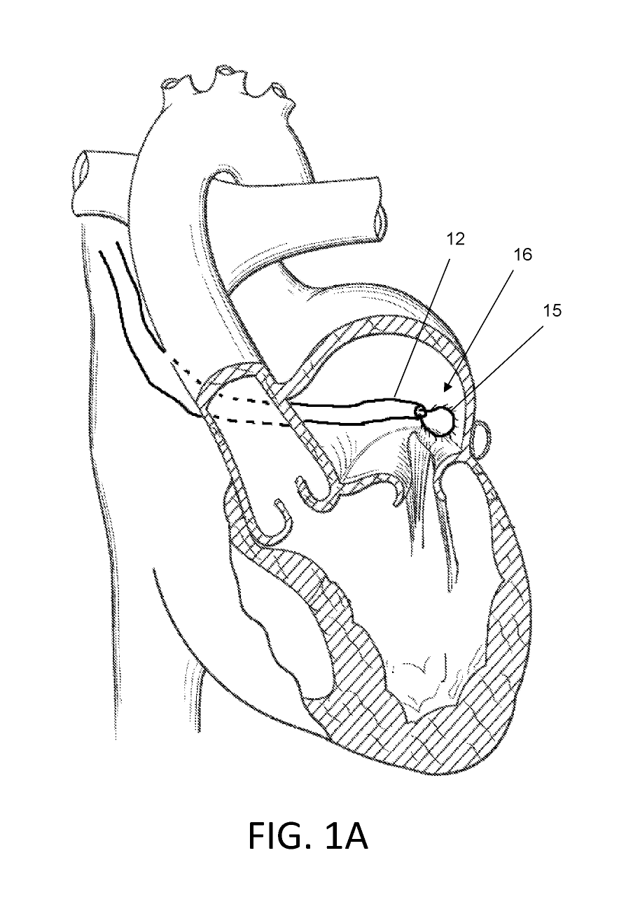

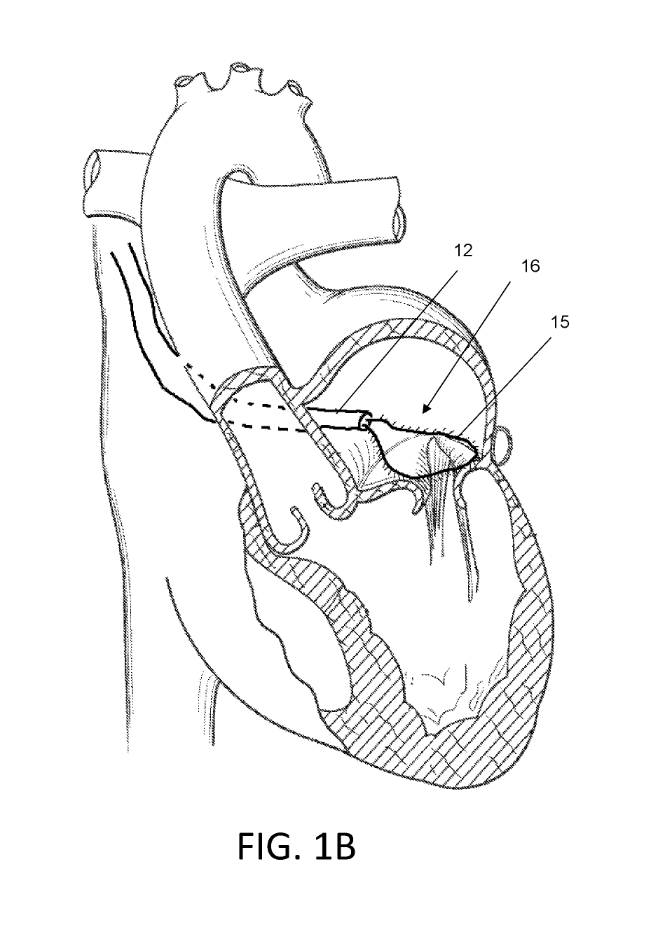

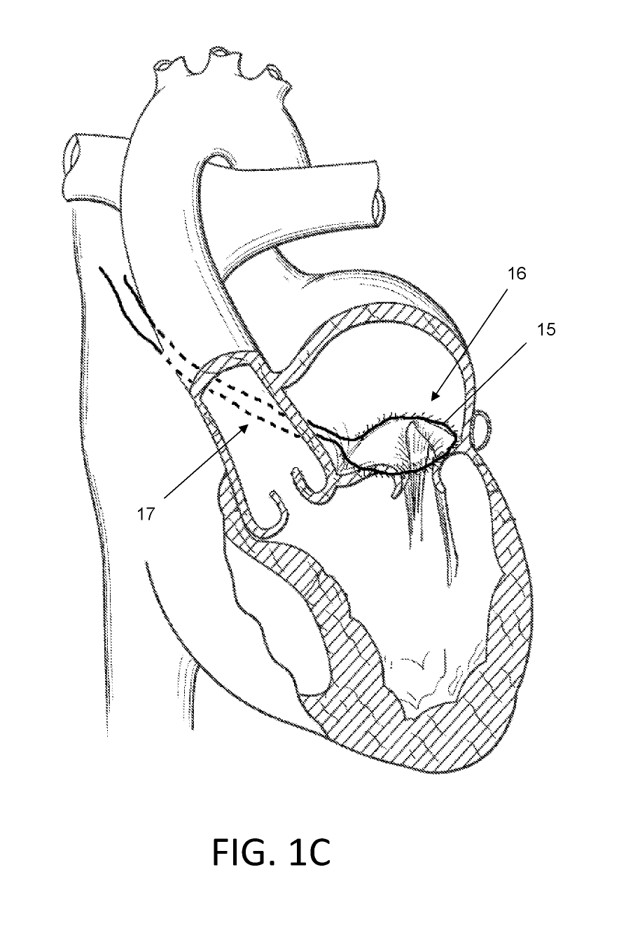

FIGS. 1A-1C depicts a first approach for installing a cinching mechanism onto a cardiac valve annulus at successive stages of deployment. In this embodiment, a ring 15 is delivered via a catheter 12 onto the annulus. The ring 15 is pushed out distally beyond the distal end of the catheter 12. The external surface of the ring 15 has a plurality of tiny whisker-like barbs 16 disposed thereon, and the barbs 16 are configured to engage the tissue due to dispensing contact pressure and hold the ring 15 in place until tissue ingrowth strengthens the bond between the ring 15 and the annulus. The barbs 16 are formed of a biocompatible material (e.g., stainless steel) configured to penetrate the tissue of the annulus and resist withdrawal from the tissue. In some embodiments, the shape of the ring 15 is pre-formed to match the shape of the annulus onto which it will be installed. In some embodiments, the ring is formed from a shape-memory material such as nitinol that automatically returns to a predetermined shape after it exits the distal end of the catheter.

The ring 15 is pushed out distally beyond the distal end of the catheter 12, as seen in FIG. 1A and is pressed against the annulus (e.g., by manipulating a set of controls dispose at the proximal end of the catheter 12, not shown) until the barbs 16 on that portion of the ring 15 become embedded into the annulus. The ring 15 is subsequently pushed further distally, until the entire ring 15 moves distally beyond the distal end of the catheter 12, as seen in FIG. 1B. Manipulation of the ring 15 continues until the entire ring 15 has been pressed against the annulus and the barbs 16 have engaged the entire annulus, as seen in FIG. 1C. A set of tethers 17 are slidably connected to the ring 15. In some embodiments, the ring 15 has a hollow core and the tethers 17 run through the hollow core. In alternative embodiments, other sliding arrangements are used. After the entire ring 15 has been pressed against the annulus so that the barbs 16 engage the entire annulus, the catheter is withdrawn, and the ring 15 and the tethers 17 are left behind, as seen in FIG. 1C.

In some embodiments, the external surface of the ring 15 comprises a coating or sleeve that promotes tissue ingrowth. Note that the initial attachment strength of the barbs 16 into the annulus does not have to be high enough to withstand cinching. Instead, the initial attachment strength need only be high enough to withstand the motion associated with the beating of the heart. As long as the initial attachment strength is high enough to withstand this motion, tissue ingrowth will strengthen the bond between the annulus and the ring 15, typically over the course of 1-3 months. After the bond has been sufficiently strengthened, the ring 15 can be cinched by accessing the tethers 17 in one or more subsequent procedures.

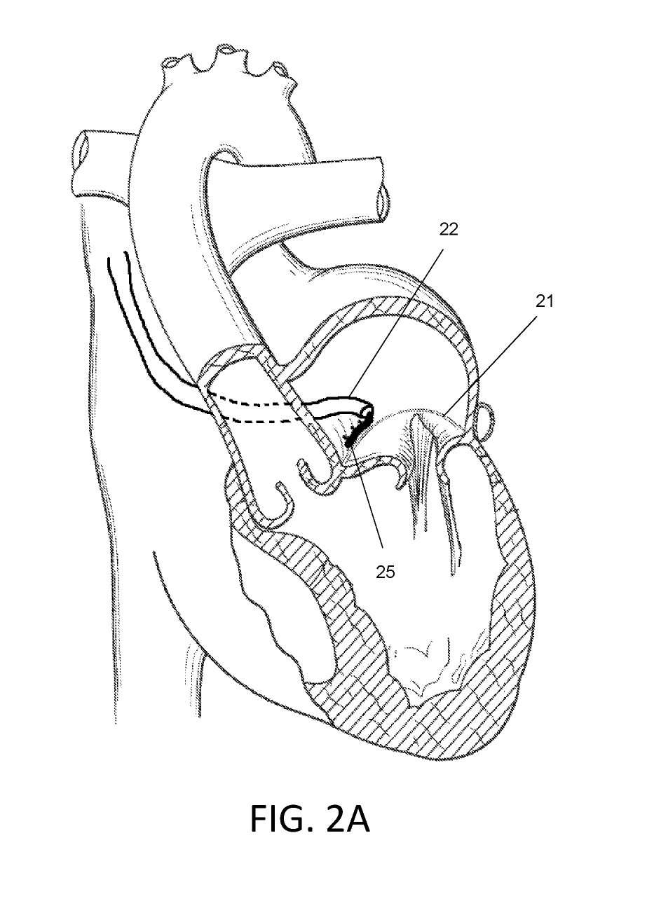

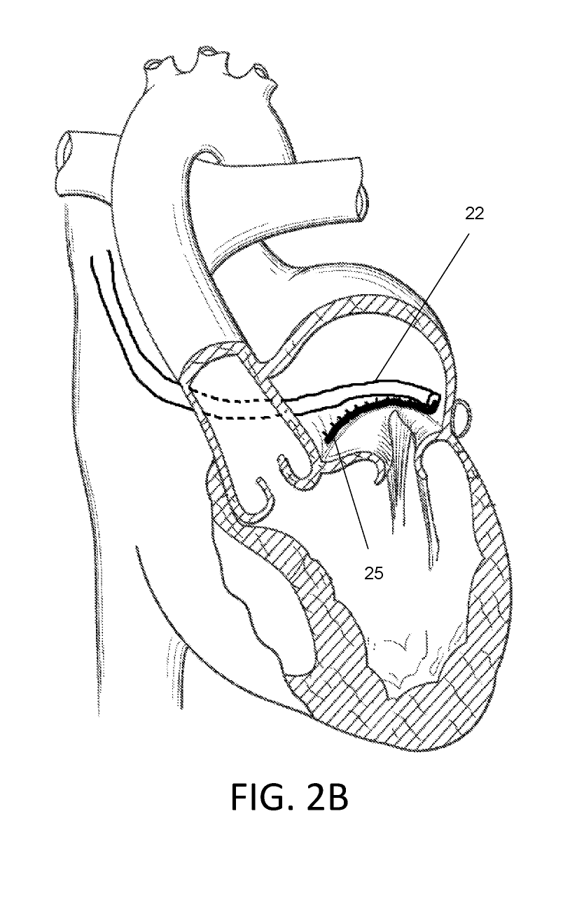

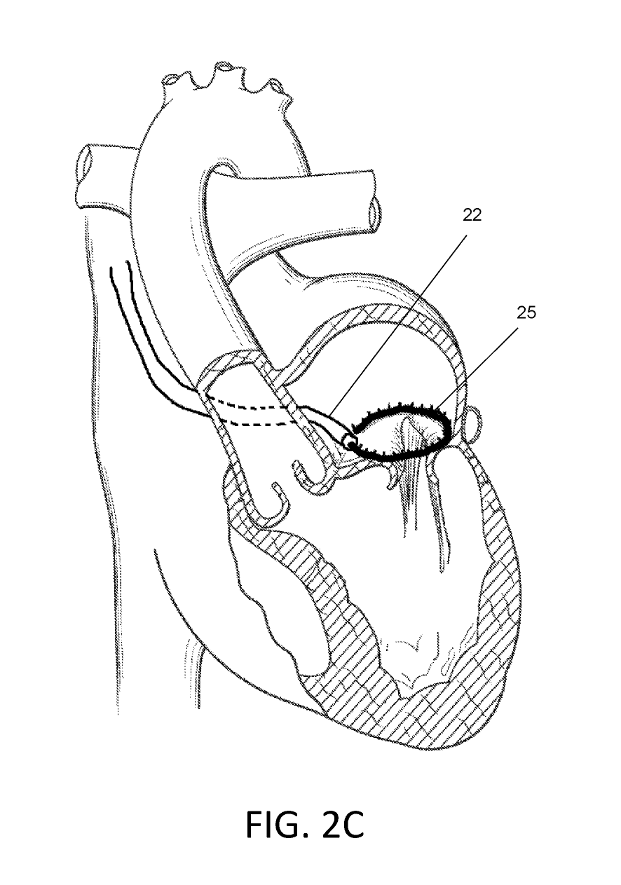

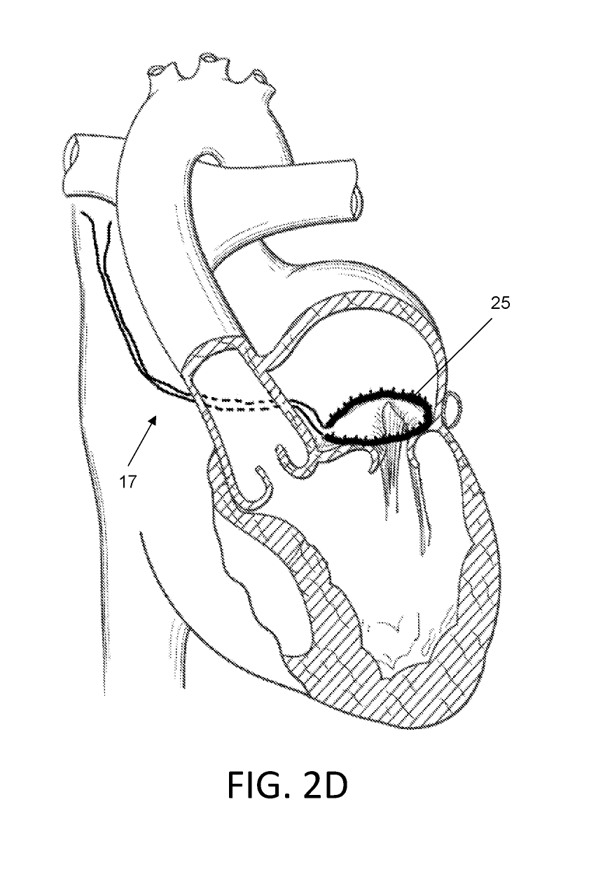

FIGS. 2A-D depicts another embodiment of a cinching mechanism that is installed on a cardiac valve annulus in successive stages of deployment. This embodiment is similar to the FIG. 1 embodiment, except that instead of beginning with a pre-formed ring that is pushed out of the end of the catheter, this embodiment begins with an unformed ring 25 that is delivered via a catheter 22, and the ring is formed in place. The distal end of the unformed ring 25 is extended beyond the distal end of the catheter 22 as depicted in FIG. 2A, and affixed to the annulus using any of a variety of approaches. In some embodiments, the distal end of the unformed ring 25 is attached using sutures or anchors. In alternative embodiments, it can be attached using barbed whiskers similar to those described above in connection with FIG. 1 and pressing those whiskers against the tissue of the annulus. Additional portions of the unformed ring 25 are sequentially extended distally beyond the distal end of the catheter 22, as depicted in FIG. 2B, and those additional portions of the unformed ring 25 are sequentially attached to the annulus. This process continues until the entire unformed ring 25 has been extended distally beyond the distal end of the catheter 22 and attached to the annulus, as depicted in FIG. 2C.

After the entire unformed ring 25 has been attached to the annulus, a pair of tethers 27 are positioned with the respect to the ring 25 so that the tethers 27 can be used to cinch the ring 25. One suitable approach for positioning the tethers 27 with respect to the ring 25 is to use a ring 25 that has a hollow core. The tethers 27 can be pre-threaded through the hollow core before the ring 25 is extended distally beyond the distal end of the catheter 22. But in this case, the tethers 27 initially do not extend beyond the distal end of the unformed ring 25. Instead the tethers 27 terminate at a button or other protrusion that sits just beyond the distal end of the ring 25. After the unformed ring 25 has been laid out and positioned in the configuration of a ring, the button at the distal end of the tethers 27 is grasped and pulled out from the distal end of the unformed ring 25 and pulled in a proximal direction back through the catheter 22, until the portion of the tethers 27 that originally resided at the distal end of the unformed ring 25 has moved proximally through the catheter 22. At this point, both ends of the tethers 27 will be positioned back at the proximal end of the catheter 22. The catheter can then be withdrawn, leaving the tethers 27 in position, as depicted in FIG. 2D.

In some embodiments, the external surface of the ring 25 comprises a coating or sleeve that promotes tissue ingrowth. As described above in connection with the FIG. 1 embodiment, the initial attachment strength of the ring 25 into the annulus does not have to be high enough to withstand cinching, and tissue ingrowth is used to strengthen the bond between the annulus and the ring 25. After the bond has been sufficiently strengthened, the ring 25 can be cinched by accessing the tethers 27 in one or more subsequent procedures.

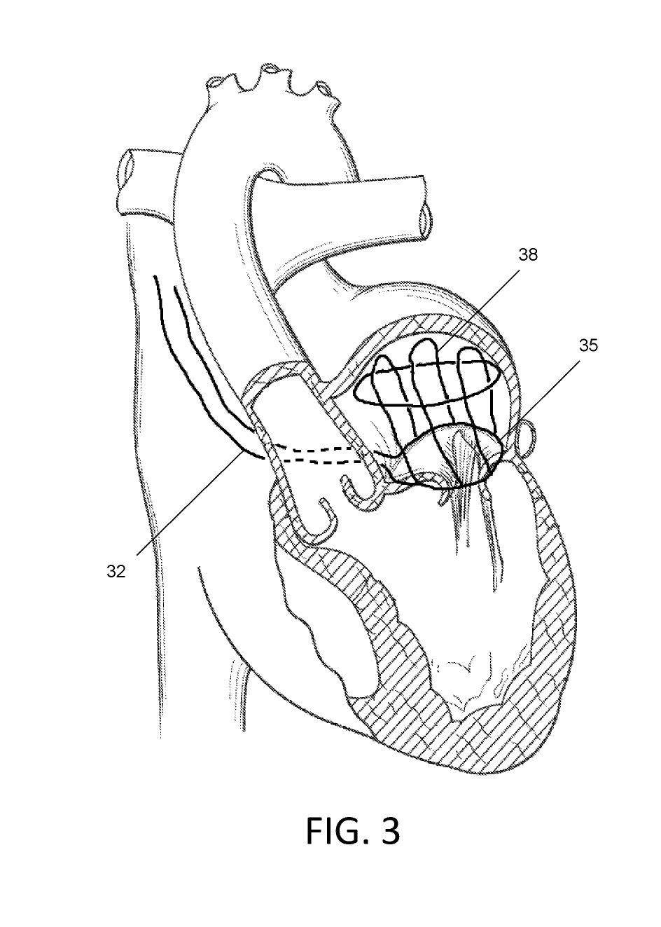

FIG. 3 depicts an alternative embodiment in which a cinching ring 35 is delivered to a valve annulus via a catheter 32. The cinching ring 35 is suspended in position on the annulus by a dome frame 38 that is expanded against the atrium wall. The dome frame 38 is preferably made from a biocompatible springy material such as stainless steel or nitinol. Both the cinching ring 35 and the dome frame 38 are delivered in a collapsed configuration via the catheter 32, and are expanded into the shape depicted in FIG. 3 after the distal tip of the catheter 32 has been extended into the atrium. A set of tethers (not shown) similar to those described above in connection with the FIGS. 1 and 2 embodiments are connected to the cinching ring 35. After the cinching ring 35 is installed in position in the annulus, the bond between the cinching ring 35 and the annulus is strengthened by tissue healing. After the tissue healing has strengthened the bond to a sufficient degree, the cinching ring 35 can be cinched, as described above in connection with the FIGS. 1 and 2 embodiments.

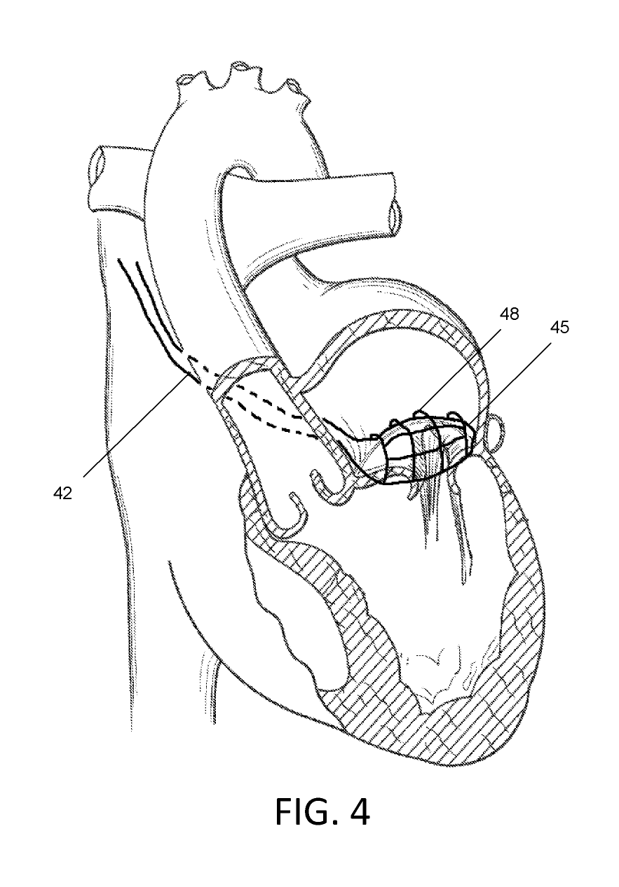

FIG. 4 depicts an alternative embodiment in which a cinching ring 45 is delivered to a valve annulus via a catheter 42. The cinching ring 45 is suspended in position on the annulus by a flexible spring dome 48 that maintains the cinching ring 45 in an expanded configuration. The spring dome 48 is preferably made from a biocompatible springy material such as stainless steel or nitinol. Both the cinching ring 45 and the spring dome 48 are delivered in a collapsed configuration via the catheter 42, and are expanded into the shape depicted in FIG. 4 after the distal tip of the catheter 42 has been extended into the atrium. A set of tethers (not shown) similar to those described above in connection with the FIGS. 1 and 2 embodiments are connected to the cinching ring 45. After the cinching ring 45 is installed in position in the annulus, the bond between the cinching ring 45 and the annulus is strengthened by tissue healing. After the tissue healing has strengthened the bond to a sufficient degree, the cinching ring 45 can be cinched, as described above in connection with the FIGS. 1 and 2 embodiments.

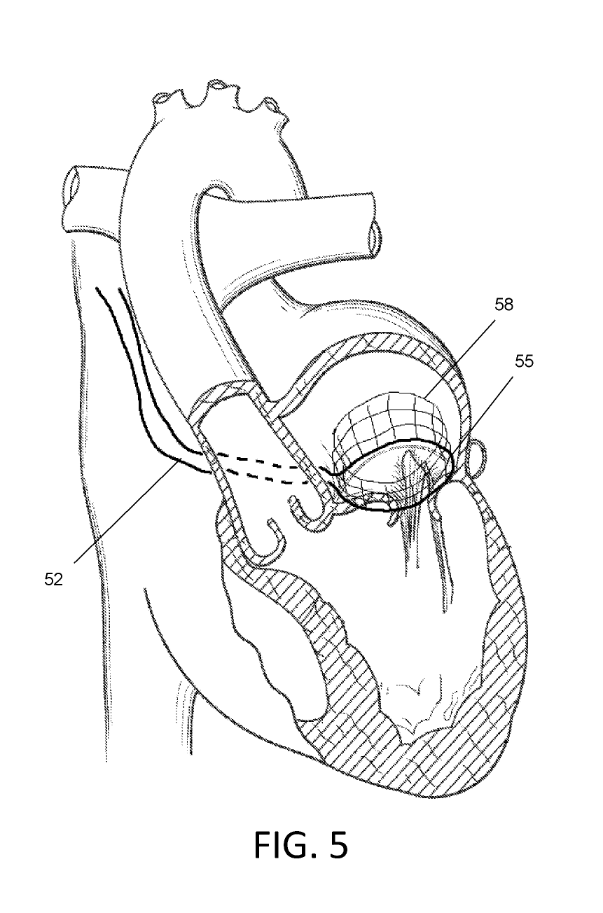

FIG. 5 depicts an alternative embodiment in which a cinching ring 55 is delivered to a valve annulus via a catheter 52. The cinching ring 55 is suspended in place by a large thin stent 58 that maintains the cinching ring 55 in an expanded configuration to create pressure against the atrium wall. The stent 58 is preferably made from a biocompatible springy material such as stainless steel or nitinol. Both the cinching ring 55 and the stent 58 are delivered in a collapsed configuration via the catheter 52, and are expanded into the shape depicted in FIG. 5 after the distal tip of the catheter 52 has been extended into the atrium. A set of tethers (not shown) similar to those described above in connection with the FIGS. 1 and 2 embodiments are connected to the cinching ring 55. After the cinching ring 55 is installed in position in the annulus, the bond between the cinching ring 55 and the annulus is strengthened by tissue healing. After the tissue healing has strengthened the bond to a sufficient degree, the cinching ring 55 can be cinched, as described above in connection with the FIGS. 1 and 2 embodiments.

Note that in the embodiments described above in connection with FIGS. 1-5, the installation of the cinching mechanism is depicted in the context of the mitral valve annulus. But these embodiments are not limited to the mitral valve annulus, and any of the embodiments described above can be modified for installation onto a different cardiac valve annulus (e.g. the tricuspid valve annulus) by making appropriate adjustments to the dimensions and delivery systems, as will be appreciated by persons skilled in the relevant arts.

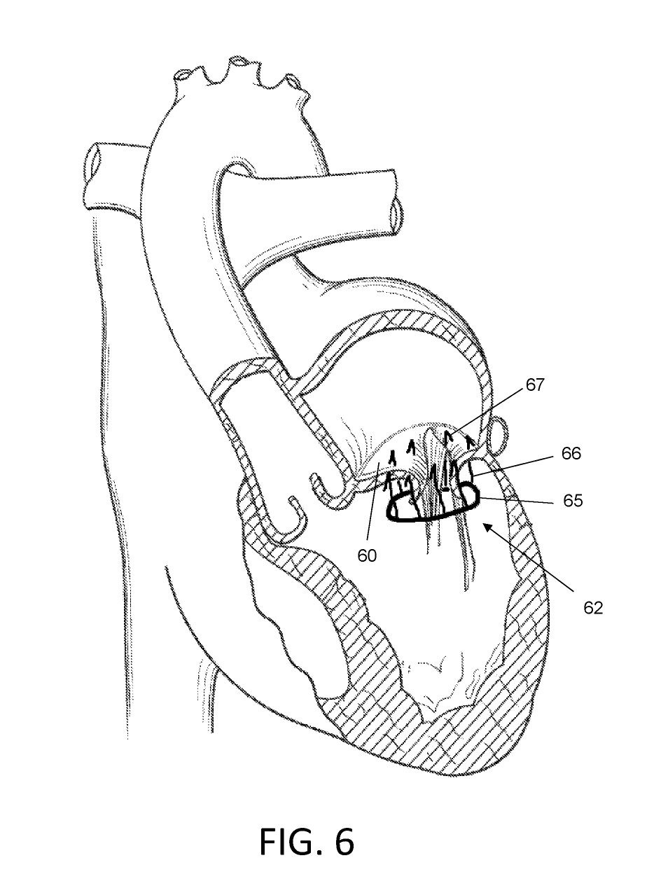

FIG. 6 depicts one embodiment of an annulus ring 62 that has been installed onto the mitral valve annulus. This annulus ring 62 is useful to prevent the annulus from expanding and/or to serve as a dock into which a replacement valve can be installed. The annulus ring 62 includes a ring portion 65 and a plurality of support rods 66 that extend perpendicular to the plane of the ring portion 65. Each of the support rods 66 has a barb 67 at the distal end of the respective support rod 66. Note that while FIG. 6 depicts eight support rods 66, a different number of support rods can be used (e.g., between 4 and 16 support rods).

In some embodiments, the ring portion 65 is made of a biocompatible metal such as stainless steel or nitinol. In alternative embodiments, the ring portion 65 may be implemented using flexible cord with a high tensile strength such as Dyneema®. Optionally, the ring portion 65 may be surrounded by one or more sleeves of a material that accepts tissue ingrowth. The support rods 66 and the barb 67 are preferably made from a biocompatible metal such as stainless steel. The length of the support rods 66 may be varied as required by the relevant anatomy.

The annulus ring 62 is installed into the leaflets 60 of the valve by delivering the annulus ring 62 into the left ventricle in a collapsed state via a catheter (not shown) and subsequently expanding the annulus ring 62 into the shape depicted in FIG. 6 after it has exited the distal end of the catheter. Subsequently, the support rods 66 are pressed into the tissue of the leaflets 60 via appropriate catheter-based controls so that the barbs 67 pierce the leaflets 60. At this point, the ring portion 65 is suspended under the leaflets by the support rods 66, and the support rods 66 combined with the barb 67 prevent the annulus ring 62 from falling into the ventricle until such time that a valve can be installed into the annulus ring 62 (e.g. 10 to 50 minutes after deployment of the annulus ring 62).

The support rods 66 are preferably relatively long (e.g., at least 1 cm) and flexible. The relatively loose connection and the flexibility of the support rods allows the valve leaflets to move relatively freely after the annulus ring 62 has been installed and keep functioning until such time that a valve is deployed into the ring. Notably, the plurality of support rods 66 point from the ventricle to the atrium so after delivering the valve the pressure differential will enhance the anchoring. More specifically, the valve will not be able to slide off the annulus ring 62. In addition, the annulus ring 62 will not be able to “jump” over the leaflets because the annulus ring 62 will always be always pushed up (i.e. in the direction of the left atrium) by the forces inherent in a beating heart. As a result, the whole assembly will stay in place, rocking up and down with the motion of the leaflets.

Once the support rods 66 have been attached to the leaflets 60, a replacement valve (not shown) can be delivered via a catheter (not shown) in a collapsed state to the interior of the annulus ring 62. After reaching this position, the replacement valve is expanded. The annulus ring 62 will prevent the replacement valve from expanding too far, and will also anchor the replacement valve in position so that it will not be dislodged by the normal operation of the beating heart. Alternatively, the annulus ring 62 may be used without a replacement valve.

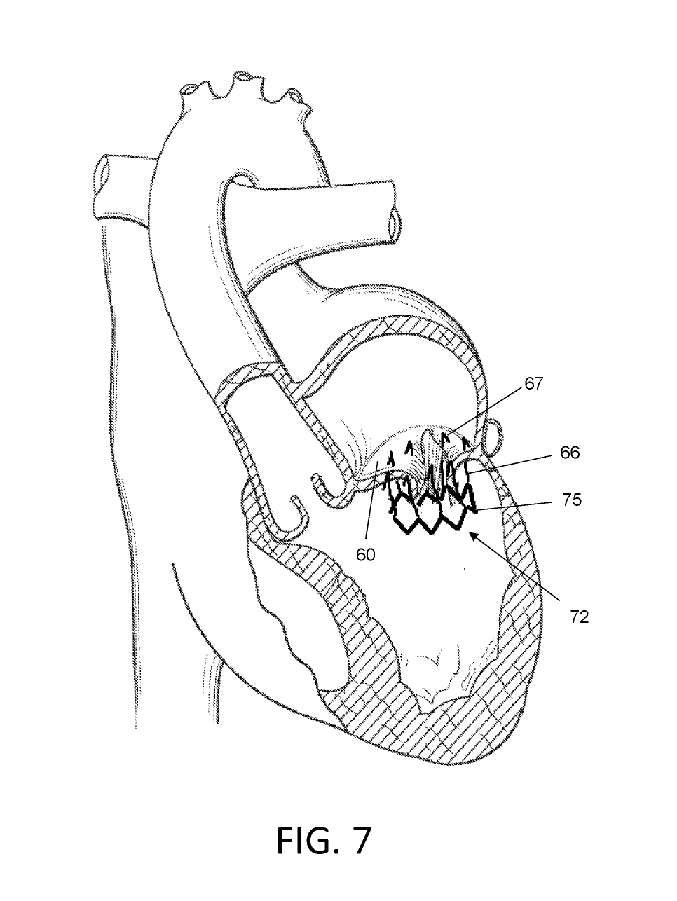

FIG. 7 depicts a variation of the FIG. 6 embodiment, and corresponding reference numbers refer to the corresponding features described above in connection with FIG. 6. The annulus ring 72 of the FIG. 7 embodiment is similar to the annulus ring 62 of the FIG. 6 embodiment, except that the ring portion 75 in the FIG. 7 embodiment is formed from a zigzag shaped expandable structure. This annulus ring 72 is also useful to prevent the annulus from expanding and/or to serve as a dock into which a replacement valve can be installed. The use of the zigzag shaped structure for forming the ring portion 75 improves the collapsibility of the annulus ring 72 to facilitate its delivery to its ultimate destination via a catheter (not shown). Once the annulus ring 72 has exited the distal end of the catheter, it will expand into the position depicted in FIG. 7.

Once the support rods 66 have been attached to the leaflets 60, a replacement valve (not shown) can be delivered via a catheter (not shown) in a collapsed state to the interior of the annulus ring 72. After reaching this position, the replacement valve is expanded. The annulus ring 72 will expand until the zigzags in the ring portion 75 have straightened out. But after that, the annulus ring 72 will prevent the replacement valve from expanding any further, and will also anchor the replacement valve in position so that it will not be dislodged by the normal operation of the beating heart.

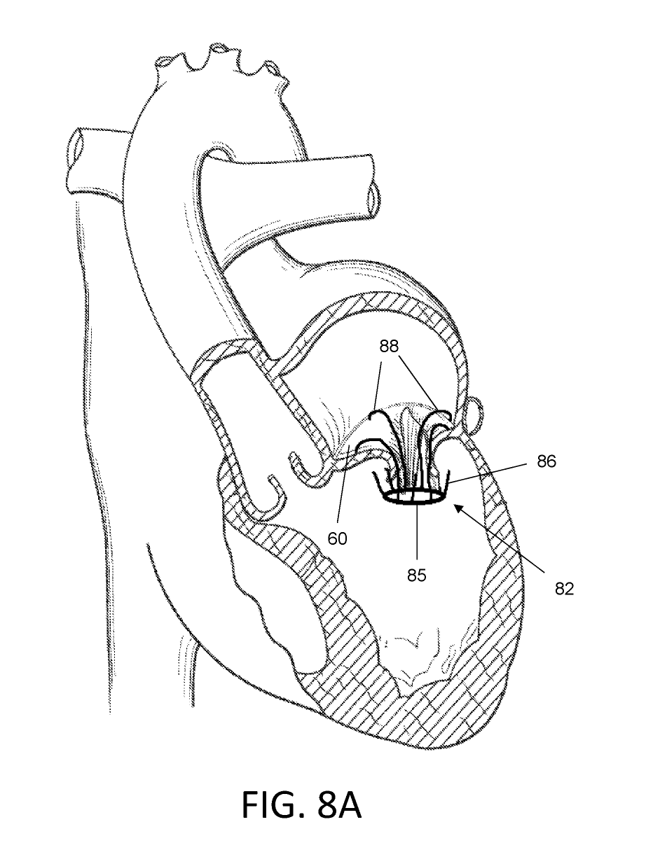

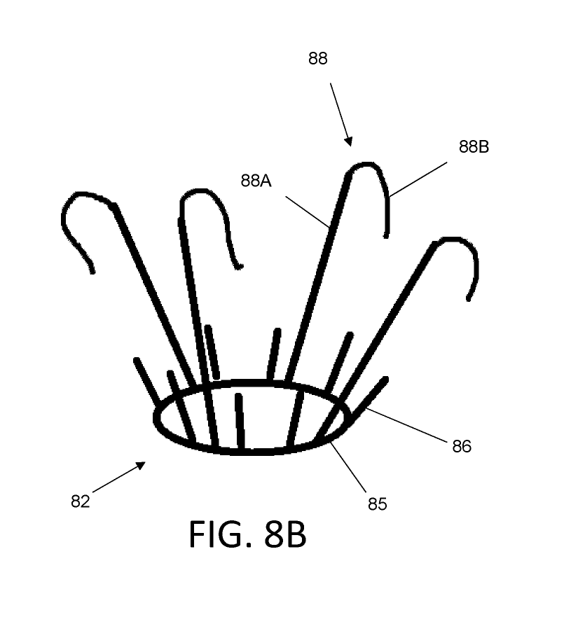

FIG. 8A depicts another embodiment of an annulus ring 82 that has been installed onto the mitral valve annulus. This embodiment is similar to the FIG. 6 embodiment, with the ring portion 85 and the support rods 86 corresponding, respectively, to reference numbers 65 and 66 described in connection with FIG. 6 above. The distal end of each of the support rods 86 has a barb (not shown) that is similar to the barbs 67 of the FIG. 6 embodiment. These barbs are directed upwards from the left ventricle towards the left atrium so that once the barbs have passed into the leaflets 60 from the left ventricle side, they cannot be pulled out in the reverse direction.

But in addition to these features that are similar to the FIG. 6 embodiment, the annulus ring 82 of the FIG. 8A embodiment also includes a plurality of support arms 88 that are configured to suspend the ring portion 85 within the left ventricle beneath the leaflets 60, as depicted in FIG. 8A. These support arms 88 have an extension portion 88A and a curved retainer portion 88B that curves radially outward, as depicted in FIG. 8B. Note that while FIGS. 8A and 8B depicts four support arms 88, a different number of support arms may be used (e.g. between 3 and 8). The annulus ring 82 is maneuvered into the position depicted in FIG. 8A (e.g., using one of the approaches described below). In this position, the support arms 88 hold the annulus ring 82 loosely in the position depicted in FIG. 8A, until such time that a replacement valve can be installed within the ring portion 85 of the annulus ring 82. During the valve installation procedure, a replacement valve (not shown) is expanded within the ring portion 85 and the barbs at the distal ends of the support rods 86 are pressed into the leaflets 60, which fixes the replacement valve at an appropriate position between the left ventricle and the left atrium. The support rods 86 in this embodiment provide the same functionality as a support rod 66 in the FIG. 6 embodiment discussed above.

The annulus ring 82 of the FIG. 8A/B embodiment can be maneuvered into the position depicted in FIG. 8A from the ventricle side by maneuvering the distal end of the catheter (not shown) into the left ventricle, and then passing the distal end of the catheter into the left atrium. For this approach, the annulus ring 82 is collapsed within the catheter so that the curved retainer portions 88B are disposed distally with respect to the extension portions 88A. After the distal end of the catheter has passed into the left atrium, the curved retainer portions 88B are extended out past the distal end of the catheter. The support arms 88 are preferably configured to expand radially outward as soon as they have exited the distal end of the catheter. The outer sleeve of the catheter can then be withdrawn, and the expanded curved retainer portions 88B will prevent those portions from traveling backwards into the left ventricle. When the outer sleeve of the catheter is completely withdrawn, the ring portion 85 will be disposed in the left ventricle, as depicted in FIG. 8A.

Alternatively, the annulus ring 82 of the FIG. 8A/B embodiment can be maneuvered into the position depicted in FIG. 8A from the atrium side by first maneuvering the distal end of the catheter (not shown) into the left atrium, and then passing the distal end of the catheter into the left ventricle. For this approach, the annulus ring 82 is collapsed within the catheter so that the ring portion 85 is disposed distally within the catheter with respect to the curved retainer portions 88B. After the distal end of the catheter has passed into the left ventricle, the ring portion 85 is extended out past the distal end of the catheter. The outer sleeve of the catheter is then withdrawn while the annulus ring 82 is held at a fixed axial position. When the outer sleeve is sufficiently withdrawn, the curved retainer portions 88B will expand within the left atrium, resulting once again in the configuration depicted in FIG. 8A.

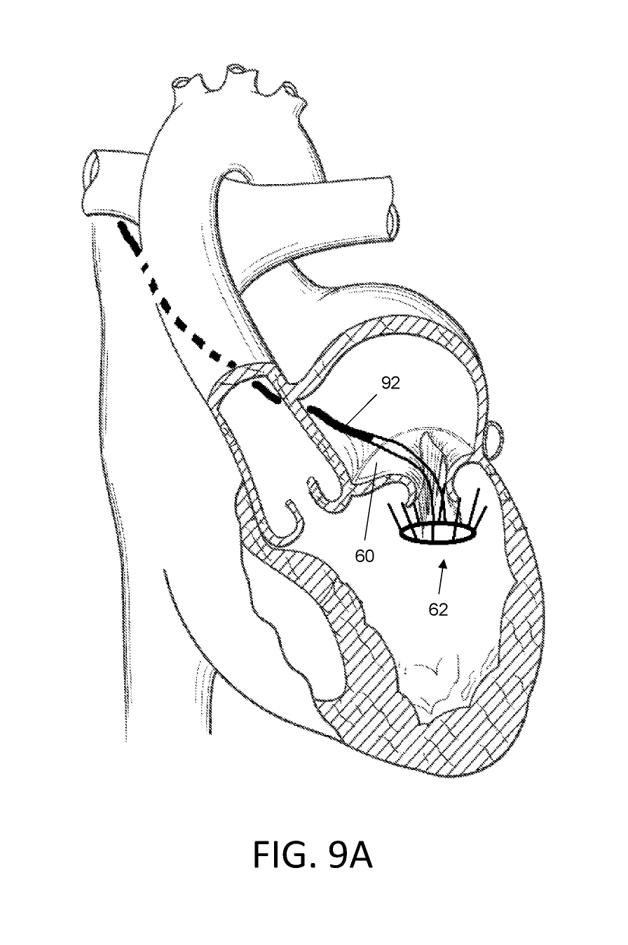

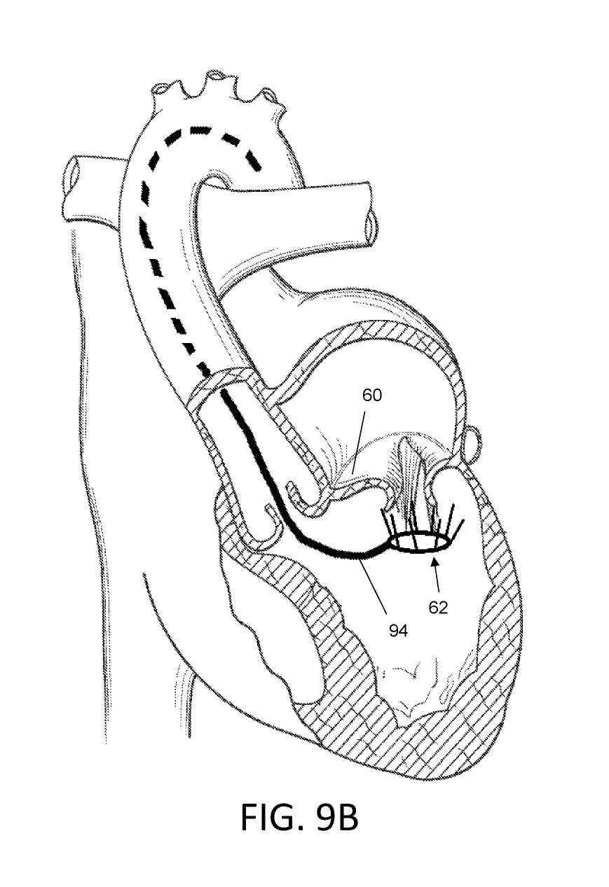

FIG. 9A depicts an alternative approach for loosely positioning an annulus ring 62 in the vicinity of the mitral valve until such time that a replacement valve is ready to be installed. The annulus ring 62 in this embodiment is similar to the correspondingly numbered annulus ring 62 in the FIG. 6 embodiment discussed above. In this embodiment, annulus ring 62 is suspended under the leaflets by a catheter 92 that is extended from the native valve inlet side to the vascular system. The catheter 92 can either be the same catheter that is used to deliver the annulus ring 62 or a supplemental catheter that is introduced for the sole purpose of temporarily holding the annulus ring 62 in position. In either case, the annulus ring 62 is held in position by the operator using the catheter 92 until a replacement valve (not shown) can be installed onto the annulus ring 62 in a subsequent procedure (which will typically be between 10 and 50 minutes after the annulus ring 62 has been delivered). The replacement valve can either be delivered by the same catheter that was used to deliver the annulus ring 62 or a different catheter. As explained above in connection with the FIG. 6 embodiment, the annulus ring 62 has barbs at the distal end of the support rods that are directed upwards from the left ventricle towards the left atrium. After a replacement valve is deployed inside the annulus ring 62 and the barbs are driven into the leaflets 60 from below, the temporary catheter 92 is removed.

FIG. 9B depicts another alternative approach for loosely positioning an annulus ring 62 in the vicinity of the mitral valve until such time that a replacement valve is ready to be installed. This approach is similar to the FIG. 9A embodiment discussed above (and corresponding numbers refer to corresponding features), except that a different position for the temporary catheter is used. More specifically, in this embodiment, the temporary catheter 94 extends from the native valve outlet side to the vascular system via the aorta. Here again, the catheter is removed after the valve is deployed.

Note that in the embodiments described above in connection with FIGS. 6-9, the installation of the annulus ring is depicted in the context of the mitral valve annulus. But these embodiments are not limited to the mitral valve annulus, and any of the embodiments described above can be modified for installing an annulus ring onto a different cardiac valve annulus (e.g. the tricuspid valve annulus) by making appropriate adjustments to the dimensions and delivery systems, as will be appreciated by persons skilled in the relevant arts.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for cinching a cardiac valve annulus, the apparatus comprising:
a catheter;
a ring of material configured for delivery to the vicinity of a cardiac valve annulus via the catheter, the ring having an external surface; and
a plurality of whisker-like barbs disposed on the external surface of the ring, wherein the barbs are configured to engage tissue when pressed against the tissue, and wherein the barbs are configured to hold the ring in place until tissue ingrowth strengthens a bond between the ring and the annulus.

2. The apparatus of claim 1, wherein the shape of the ring is pre-formed to match the shape of the annulus onto which it will be installed.

3. The apparatus of claim 1, wherein the ring is formed from a shape-memory material that automatically returns to a predetermined shape after the ring exits the catheter.

4. The apparatus of claim 1, wherein the ring has a hollow core, and wherein the apparatus further comprises a tether that runs through the hollow core.

5. The apparatus of claim 1, wherein the external surface of the ring includes a coating that promotes tissue ingrowth.
